(19) United States
(12) Patent Application Publication
(10) Pub. No.: US 2026/0345925 A1
(43) Pub. Date: Nov. 5, 2026

(54) METHOD FOR MIXING A FIRST AND A SECOND PHARMACEUTICAL POWDERY SUBSTANCE AND USE OF A CONTAINER WITH TWO CHAMBERS IN AN INHALATION DEVICE

(71) Applicant: Alfred VON SCHUCKMANN, Kevelaer (DE)

(72) Inventor: Alfred VON SCHUCKMANN, Kevelaer (DE)

(21) Appl. No.: 19/488,876

(22) PCT Filed: May 27, 2024
(86) PCT No.: PCT/EP2024/064531

§ 371 Date: Dec. 1, 2025

(30) Foreign Application Priority Data
Jun. 2, 2023 (DE) 10 2023 114 581.9

(51) Int. Cl. A61M 15/00

(52) U.S. Cl. CPC A61M 15/0003; A61M 15/0008; A61M 15/0035; A61M 2202/064

(57) ABSTRACT

A method for mixing a first and a second pharmaceutical powdery substance in an inhalation device with which pharmaceutical substance is transported by a suction of air, wherein the first and the second substance are contained in a container with two separate chambers, wherein further the chambers are arranged one behind the other along a longitudinal axis of the container. In a first step the chambers of the container are opened by piercing, with forming piercing openings, wherein the opening by piercing is carried out with respect to the longitudinal axis on opposite sides. In a further step the container is rotated about an axis of rotation in the course of suction in a receiving chamber of the inhalation device, which runs transversely to the longitudinal axis, whereby the substances from each chamber are discharged radially outwards from the container and a mixing of the two substances thus discharged is achieved.

### FIELD OF TECHNOLOGY

The invention relates to a method for mixing a first and a second pharmaceutical powdery substance in an inhalation device with which pharmaceutical substance is transported by a suction of air, wherein the first and the second substance are contained in a container with two separate chambers, wherein further the chambers are arranged one behind the other along a longitudinal axis of the container, wherein moreover in a first step the chambers of the container are opened by piercing, with forming piercing openings, wherein the opening by piercing is carried out with respect to the longitudinal axis on opposite sides.

The invention further relates to the Use of a container with two chambers, a first or second chamber, in an inhalation device, the chambers being filled with a first and a second pharmaceutical powdery substance respectively, the inhalation device is having piercing needles for piercing each chamber of the container, the first chamber further is having a first base and the second chamber is having a second base, the container further having a partition, the partition separating the first chamber from the second chamber, wherein in addition the partition forms the first and second base (and the first and second chambers extend in opposite directions to one another, starting from the first and second base, respectively, along a common longitudinal axis of the container and form a peripheral wall and a ceiling area opposite the base, each ceiling area being concavely curved and being pierceable, for the delivery of the first and second substances from the container, by rotating the container about an axis of rotation running transversely to the longitudinal axis.

### STATE OF THE ART

Containers containing pharmaceutical powdery substances are known, in particular in the form of capsule-like containers, the contents of which are used in particular for inhalation by using of an inhalation device. Such containers are usually provided with openings immediately before an inhalation process is carried out, through which the substance can emerge from the container into the suction flow that results during the inhalation process. This suction flow is usually achieved solely as a result of the person using the inhaler taking a deep breath.

To open the container, the inhalation device has piercing needles, which can be moved from a basic position into an activation position passing through the wall of the container by the user's willful actuation. The resulting openings in the container are made on the opposite sides of the container with respect to the longitudinal axis. For example, such an inhalation device is known from EP 1 270 034 B1 (U.S. Pat. No. 7,284,552 B2).

Other containers are known which have separate chambers arranged one behind the other in the direction of a longitudinal axis of the container. In this regard, reference is made to WO 2020/229 852 A1 (US 2022/0 213 685 A1). By arranging two separate chambers in the container, two substances can be accommodated in the container, which are only mixed into an active ingredient mixture by the suction flow during the discharge from the container and/or during the inhalation process.

### SUMMARY OF THE INVENTION

The object of the invention is to provide a method and a use which enable an advantageous mixing of the two substances to be achieved with the inhalation device advantageously designed.

This object is solved in view of the method in that the container in a further step, in the course of suction of air by an user, is rotated about an axis of rotation in the course of suction in a receiving chamber of the inhalation device, which runs transversely to the longitudinal axis, whereby the substances from each chamber are discharged radially outwards from the container and a mixing of the two substances thus discharged is achieved.

With regard to the use, the problem is solved by using a container with two separate chambers, whereby the chambers are arranged one behind the other along a longitudinal axis of the container, in which each chamber has a ceiling area and each ceiling area is concavely curved (from the inside), pierceable for dispensing the first and second substances from the container, by rotating the container about a transverse to the longitudinal axis running axis of rotation.

As a result of such use of a container with two separate chambers, in which a first and a second substance are received, this advantageously results—after piercing each of the chambers—only with the onset of the inhalation process, correspondingly with the start of an suction air flow initiated by the user in the course of inhalation that the two initially separated substances are mixed. According to the rotation of the container caused by the user during the suction of air about an axis of rotation directed essentially transversely to the longitudinal axis of the container as a result of the resulting centrifugal forces with respect to the first or second base, the two substances can escape. With respect to the partition wall in the direction of the longitudinal axis, substances are going to openings in the ceiling areas of the container or the chambers opposite each other respectively. The substances get conveyed outwards into the area of the receiving chamber accommodating the container and are discharged together and mixed together for inhalation via an outflow channel which preferably adjoins the receiving chamber in the direction of flow.

The substances exit the chambers preferably solely as a result of the forces occurring during the rotation of the container, in particular centrifugal forces. The partition wall, or base, provided between the chambers in the container prevents the air sucked from flowing through the container essentially in the direction of the longitudinal axis.

The ceiling areas can be like a dome—at least when viewed from the inside—whereby, due to constructive means, for example by thinning the material and/or due to the selected material from which the ceiling area in particular is made, piercing can be achieved using device-side piercing needles. There is a spherically curved ceiling—seen from the inside—which is pierced. The rotational force moves the substance towards the ceiling. Due to the curved design, the substance is then moved to the opening created by the piercing. Using the piercing needles, a piercing opening can also be created essentially in the zenith area of the respective concavely curved ceiling area or the dome-like curved ceiling area, which advantageously results in a complete emptying of the chambers and thus a complete exit due to the forces occurring in the course of rotation of the first and the second substance can be reached. The substances are going into the suction air flow.

Such an inhalation device can essentially be designed as is known from the already mentioned EP 1 270 034 B1 (U.S. Pat. No. 7,284,552 B2).

Further features are explained below, including in the description of the figures, often in their preferred association with the fundamentally described solutions or with further features. However, they can also be associated with only individual features that have already been described or with the respective further features described, or they can be of significance independently.

The piercing openings of the chambers formed by piercing can pass through overlapping paths as the container rotates about the axis of rotation. This superimposition of the paths can only be given in sections with respect to a complete rotation of the container around the axis of rotation by 360°, for example due to a slightly wobbling rotational movement of the container, so that the circumferential paths of the piercing openings may cross each other several times. The overlapping paths of the piercing openings allow a substance emerging from a piercing opening, for example the first substance emerging from a first piercing opening, to be dispensed into the circulation path of the further piercing opening leading in the direction of rotation, for example the second substance emerging from a second piercing opening. This leads to an effective mixing of the two substances already in the area of the receiving chamber, wherein this mixing maybe advantageously supported by the overlapping paths in the course of the exit and further by the suction air flow prevailing in the receiving chamber.

Due to the rotation of the container, the substances can come against a circumferential wall of the receiving chamber that extends essentially transversely to the axis of rotation and can be caught by it. Due to the centrifugal forces acting on the substances when they exit the chambers, the radial exit path of the substances with respect to the axis of rotation is limited. The substances tend to be deflected by the peripheral wall in a direction that essentially corresponds to the direction of rotation of the container, is preferably superimposed by a component acting essentially in the direction of the axis of rotation due to the suction air flow, so that essentially a helix—or spiral-like flow along and surrounding the axis of rotation—with the mixed substances can result. The substances that may come against the peripheral wall can also be distributed in the receiving chamber like a spray mist by limiting their exit path radially outwards, which can lead to further improved distribution and subsequently further improved mixing of the two substances.

The air sucked in by the user during the inhalation process can essentially flow around the container, particularly in the area of the receiving chamber. The flow can be in the circumferential direction of the peripheral wall of the receiving chamber, and possibly also in a plane transverse to the longitudinal axis of the container. The essential flow can be in the circumferential direction which may mean in the plane transverse to the longitudinal axis. An air vortex can arise that flows around the container, by means of which the escaping substance particles are mixed with one another and transported for inhalation. In the course of this transport, a further favorable mixing of the two substances that are initially separated in the container can be achieved by swirling the suction air flow, so that a largely homogeneous mixture of the two substances is supplied to the user's airway during the inhalation process. It is assumed that only a small to practically no proportion of the suction air flows into and out of a chamber or even significantly causes the escape of substance.

The receiving chamber can have a bottom with a recess that is essentially adapted to the contour of the container. In the area of this recess, according to a preferred embodiment, the user can insert the undamaged container, correspondingly having unopened chambers, in preparation for an inhalation process. More preferably, in this recess the container cannot be rotated about the axis of rotation described above. This preferably results in a defined orientation of the container to be inserted. A rotation area of the receiving chamber, in which the container can rotate about the axis of rotation extending transversely to its longitudinal axis, is formed, as it were, one floor above the depression.

According to a preferred embodiment, the piercing of the chambers is carried out when the container is in the recess of the receiving chamber. The chosen dimensions of the recess ensure that the container is guided and supported overall on the bottom and preferably on the side, so that exact perforation can be carried out reproducibly using the piercing needles to form the piercing openings sufficient exactly.

In a further embodiment, the container can be moved from the recess of the receiving chamber in the course of sucking in air in the inhalation device into the rotation area of the receiving chamber that follows in the direction of flow of the air, in which rotation area the rotation of the container only occurs due to the air flow around the axis of rotation. For this purpose, the rotation area—viewed in a plane transverse to the axis of rotation—has an extension that allows the container to rotate. Accordingly, in this regard, a substantially circular peripheral wall is preferably formed in the above-described plane, which limits the rotation range. This can also result in a diameter dimension with respect to the circumferential wall which exceeds the longitudinal dimension of the container along the longitudinal axis by a factor of, for example, 1.05 to 1.5. The height of the rotation area, viewed perpendicular to this diameter dimension, is adapted to the container width viewed perpendicular to the longitudinal axis of the container to enable free rotation of the container, and more preferably exceeds this by approximately 1.05 to 1.5 times.

The receiving chamber can accordingly essentially be composed of the area of the recess and the rotation area which forms next to a recess opening.

Each chamber of the container can have a chamber wall that is located radially outside of the chambers and is also curved with respect to an outer contour, which can correspond to the concavely curved ceiling area mentioned at the beginning. The chamber wall, which is curved on the inside, for example designed like a dome, guides the substances from both chambers to the piercing openings during the inhalation process and the associated rotation of the container around the axis of rotation. The curved inner surfaces of the ceiling areas have a funnel-like effect, particularly for substance particles sliding radially outwards along the inner wall of the chambers. The preferred arrangement of the piercing openings at the zenith of the preferably hemispherically shaped ceiling areas, in conjunction with the effect of the targeted guidance of the substance particles towards the piercing openings, supports the complete emptying of the chambers as the container rotates.

According to a preferred embodiment, the container can have three parts, a middle part which has the partition and at least a partial area of the peripheral wall, and two ceiling parts which form the ceiling areas. With regard to each chamber, this preferably results in a two-part structure. The peripheral wall can be at least partially formed in two layers due to the superimposition of sections of the middle part and the respective cover part, viewed transversely to the longitudinal axis of the container.

In one possible embodiment, one ceiling part, preferably both ceiling parts, can consist of a gelatin material. The gelatin material of the ceiling parts is shaped to be concavely curved accordingly.

All parts, namely the ceiling parts and the middle part, can also consist of a gelatin material.

In an alternative embodiment, one, several or all parts can be made of a hard plastic material. In this regard, polypropylene (PE), for example, can also be used.

If the ceiling parts in particular are made of a hard plastic material, areas thinned in terms of material thickness can be provided in the area of the piercing openings to be provided.

Preferred is an embodiment in which the middle part is made of a hard plastic material and the cover parts are made of a gelatin material. The molded parts forming the middle part and the ceiling parts can, as further preferred, be connected to one another in a form-fitting manner.

According to a possible embodiment, the peripheral wall can be designed to be cylindrical all around with respect to the longitudinal axis, further optionally, as is also preferred, with a diameter that is essentially constant in the longitudinal extent of the container. The ceiling area, which is concavely curved at each end, can, as further preferred, accommodate this diameter of the cylindrical peripheral wall essentially continuously, so that dome-like ends are formed—in an outer contour—with a radius of curvature that essentially corresponds to the radius of the cylindrical peripheral wall.

In a further embodiment, the container can have a length given in the direction of the longitudinal axis, which is greater than a width (for example diameter) of the container given perpendicular thereto. In this regard, the container can have a length which can correspond to approximately 1.5 to 5 times, further approximately 3 to 3.5 times the container width.

With regard to the disclosure, the ranges or value ranges or multiple ranges given above and below also include all intermediate values, in particular in 1/10 increments of the respective dimension, and if necessary also dimensionless. For example, the statement 1.5 to 5 times also includes the disclosure of 1.6 to 5 times, 1.5 to 4.9 times, 1.6 to 4.9 times, etc., which Revelation from 1.05 to 1.5 also the revelation from 1.15 to 1.5, 1.05 to 1.4, 1.15 to 1.4 etc. On the one hand, this revelation can be used to delimit a specified area limit from below and/or above, alternatively or additionally, also serve to reveal one or more singular values from a specified area.

### BRIEF DESCRIPTION OF THE DRAWINGS

The invention is explained below with reference to the accompanying drawing, which only represents an exemplary embodiment. The drawing shows:

FIG. 1 a perspective view of an inhalation device for carrying out the method and for using a container, in a non-use or storage position;

FIG. 2 the inhalation device according to FIG. 1, relating to a preparation position for receiving a container;

FIG. 3 the inhalation device in a perspective exploded view;

FIG. 4 the section through the inhalation device along section plane IV in FIG. 1;

FIG. 5 the section along line V-V in FIG. 4;

FIG. 6 a sectional view corresponding to FIG. 4, but after inserting a container into a receiving chamber of the inhalation device;

FIG. 7 the section along line VII-VII in FIG. 6;

FIG. 8 a subsequent illustration to FIG. 6, after the device-side piercing needles have been relocated for piercing the container;

FIG. 9 the section along line IX-IX in FIG. 8;

FIG. 10 a subsequent illustration to FIG. 8, after the piercing needles have been moved back to a basic position;

FIG. 11 a subsequent illustration to FIG. 10; after the start of a suction air flow caused by the user of the inhalation device and the associated inhalation process;

FIG. 12 the section along line XII-XII in FIG. 11;

FIG. 13 the enlargement of area XIII in FIG. 12;

FIG. 14 the container intended for use in a perspective individual view;

FIG. 15 the section along line XV-XV in FIG. 14.

### DESCRIPTION OF THE EMBODIMENTS

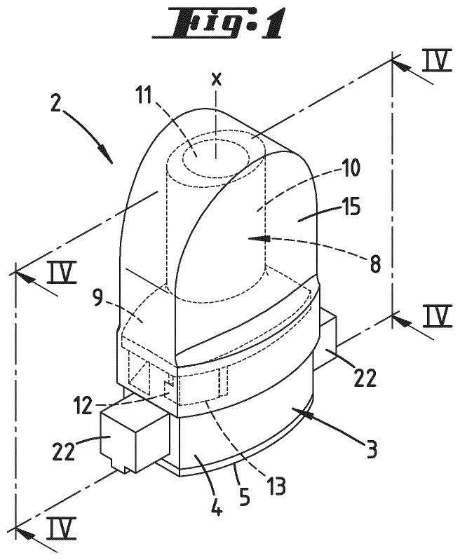

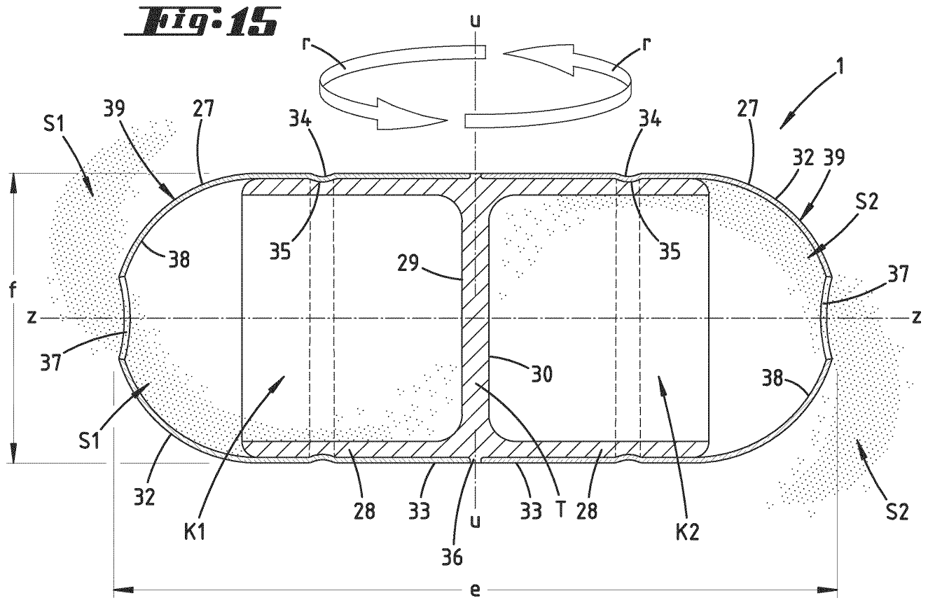

A method for mixing a first and a second pharmaceutical powdery substance S1 and S2, which are contained in two separate chambers K1 and K2 of a container 1, is shown and described. Such a container is shown, for example, in FIGS. 14 and 15. The mixing is carried out in an inhalation device 2, as shown, for example, in FIGS. 1 to 5. Described is also, and the drawings also show, an use of the container 1 in the inhalation device 2.

The inhalation device 2 initially has a housing 3, with a housing wall 4 and a housing bottom 5.

The housing wall 4 surrounds and delimits a receiving chamber 6 formed in the housing 3, which opens towards a housing edge 7 facing away from the housing bottom 5.

Assigned to the housing edge 7, the housing 3 further has a suction section 8, with a housing cover 9 which can be assigned to the housing edge 7 and is adapted to the contour of the housing edge 7, and a spout-like extension 10 which extends outwards from the housing cover 9 in the opposite direction to the housing wall 4 with a central suction channel 11.

The suction section 8 can be pivotally mounted on the housing 3 to expose the receiving chamber 6. The relevant geometric pivot axis y can extend in the direction of the spout-like extension 10. For example by pivoting the suction section 8 about the axis of rotation y, an exposure of the receiving chamber 6 is made possible, for example by pivoting the suction section 8 back, the receiving chamber 6 is closed due to a cover by the housing cover 9 of the suction section 8. In a closed position the housing cover 9 lies all around on the housing edge 7.

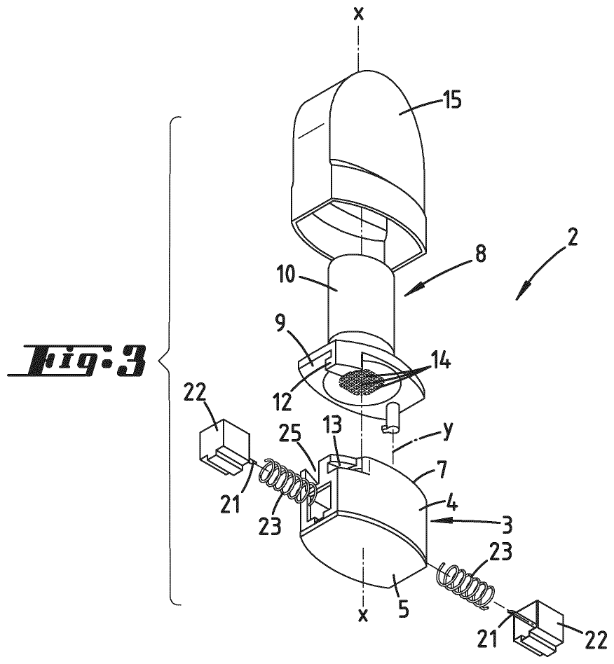

The closed position, as shown for example in FIGS. 1 and 4, can be defined by a stop limitation of the rotatability of the suction section 8. For this purpose, the suction section 8 can have a locking extension 12, which can enter a preferably shape-adapted niche 13 in the area of the housing wall 4 in a locking manner.

The region of the housing cover 9 covering the receiving chamber 6 in the closed position can be provided with an opening 14 or a plurality of openings 14. This can result in an overall grid-like opening structure in this area. As a result of these possibly several openings 14, the suction channel 11 is connected to the inside of the receiving chamber 6 in terms of flow in the chamber closed position.

The suction section 8, in particular the extension 11, can be designed for oral or nasal inhalation.

In the nonuse position of the inhalation device 2, e.g. according to FIG. 1 may, as it is also preferred, the suction section 8 be covered by a cover cap 15 which might be fixable on the housing 3 by plug on. For a preparation and for carrying out an inhalation, this cover cap 15 will be removed.

The orientation of the suction section 8, in particular the extension 11, results in a longitudinal orientation of the inhalation device 2 with a longitudinal axis x, particularly in the chamber closed position. This longitudinal axis x preferably extends in the same direction as the pivot axis y of the suction section 8, with the receiving chamber 6 preferably passing centrally through the longitudinal axis x.

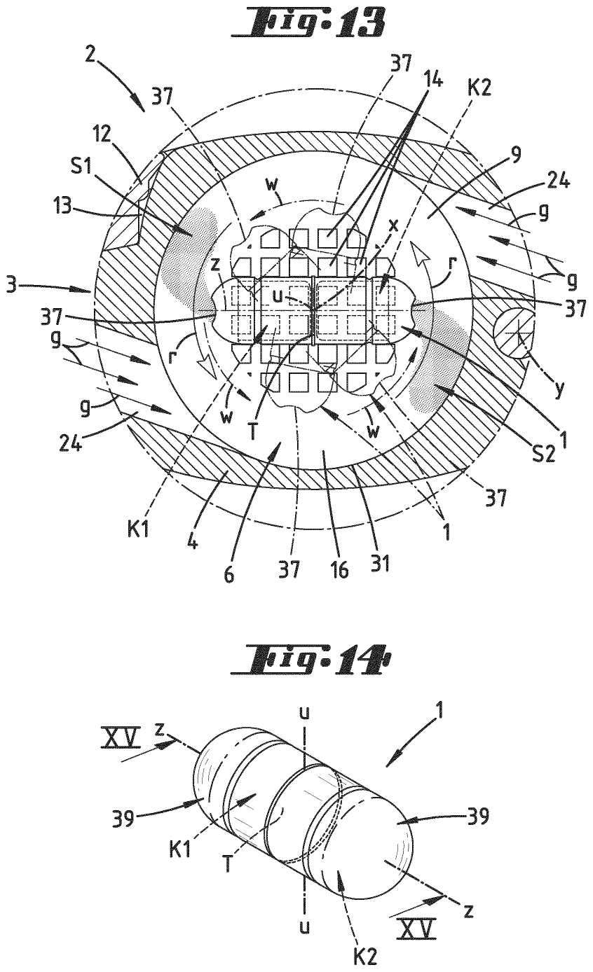

The receiving chamber 6 is essentially composed of two areas. The receiving chamber 6 initially forms a rotation area 16, with a preferably circular disk-shaped floor plan design, in which floor plan the longitudinal axis x is represented as a point (compare FIGS. 12 and 13). This results in a diameter d viewed transversely to the longitudinal axis x in the rotation area 16.

Towards the plane defined by the circumferential housing edge 7, the rotation area 16 is designed to be open (chamber opening 17), and is only covered by the housing cover 9 of the suction section 8 in the chamber closure position. Opposite this chamber opening 17, viewed in the direction of the longitudinal axis x, is a bottom 18 provided.

With reference, for example, to the illustration in FIG. 4, below the rotation area 16, correspondingly facing away from the chamber opening 17, the second area of the receiving chamber 6 results from a recess 19 starting from the bottom 18. This recess 19 is preferably positioned in the middle of the bottom 18 viewed in a direction transverse to the longitudinal axis x, a length a is chosen to be smaller than the above-described diameter d in the rotation area 16. For example, this length a can correspond to approximately 0.7 times the diameter d.

Again transverse to the length a, there is a width b of the recess 19. This can, for example, correspond to approximately 0.3 times the above-described length a of the recess 19.

In addition, viewed in the direction of the longitudinal axis x and starting from the plane of the bottom 18, there is a depth c of the recess 19, which can correspond, for example, to approximately 2 times the width b. Viewed in the same direction, there is also a depth c′ in the rotation area 16 between the bottom 18 and the plane of the chamber opening 17, which depth c′ can, for example, correspond approximately to the depth c of the recess 19.

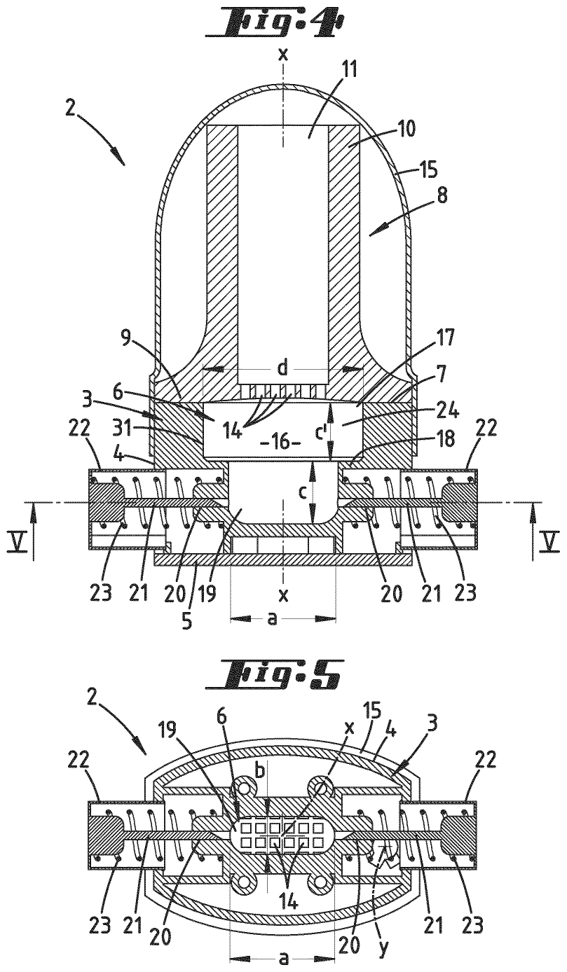

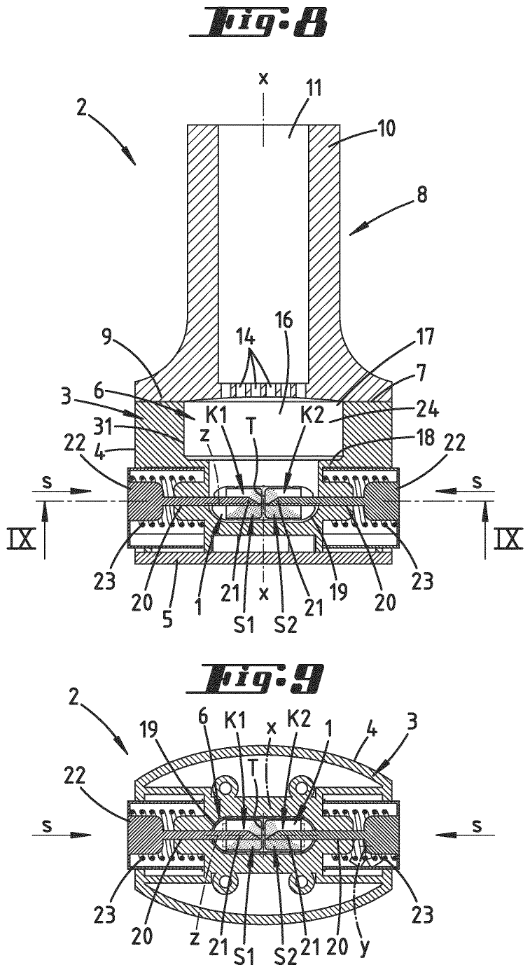

Viewed in the longitudinal extent of the recess 19, a guide 20 for a piercing needle 21 is formed at each end of the recess 19. These piercing needles 21 extend in a plane transverse to the longitudinal axis x and are further in this plane from a basic position, as shown, for example, in FIGS. 4 and 5, into a perforation position, as shown, for example, in FIGS. 8 and 9, and can be moved back to the basic position, with needle tips of the piercing needles 21 in the perforation position dipping into the space formed by the recess 19.

For this purpose, the piercing needles 21 are provided at their ends, corresponding to the receiving chamber 6 or the recess 19, with an actuation button 22 which projects freely beyond the contour of the housing 3 in the basic position. Each actuation button 22 is loaded into the basic position via a spring 23, for example a cylinder compression spring according to the illustrations. The spring 23 is supported with one end region on the housing side in the area of the guide 20 and acts on the actuation button 22 with the other end region.

The springs 23 push the actuation buttons 22 and through them the piercing needles 21 into the stop-limited basic position. A shift of the piercing needles 21 into a perforation position is only possible with will and by overcoming the restoring forces of the springs 23.

Assigned to the rotation area 16, the housing wall 4 is provided with two suction channels 24, which open towards the surroundings in opposite side areas of the housing wall 4, over which side areas the actuation buttons 22 also protrude freely (suction opening 25). As can be seen in particular from the cross-sectional illustration in FIG. 12, the suction channels 24 open into the rotation area 16 diametrically opposite with respect to the longitudinal axis x, the cross-sectional dimension and the orientation of each suction channel 24 being selected so that it is essentially in a tangential orientation to the rotation area 16, in particular to the peripheral wall 31 surrounding the rotation area 16, enters the rotation area 16.

This results in a fluidic connection between the suction channel 24, the receiving chamber 6—in particular the rotation area 16—and the suction channel 11 of the suction section 8, with both the suction channels 24 and the suction channel 11 being designed to be open to the environment.

The capsule-like container 1 is shown in detail in FIGS. 14 and 15, with the sectional view in FIG. 15 schematically illustrating the state of a substance discharge from the container 1.

The container 1 initially and essentially has a cylindrical basic contour, with two—at least seen from the inside—dome or dome-like ceiling areas 39. The container 1 can be made up of three parts, with a middle part 26 and two ceiling parts (27, 28), forming the dome-like ceiling areas 39.

The middle part 26 can essentially initially be designed like a sleeve with a sleeve wall which essentially forms at least a partial area of the essentially circular cylindrical peripheral wall 28 of the container 1. The central axis of this circular cylindrical peripheral wall 28 also forms the geometric longitudinal axis z of the container 1.

Preferably approximately in the middle of the extent of the peripheral wall 28 in the direction of the longitudinal axis z, a partition T is formed in one piece and of the same material as the peripheral wall 28. This extends in a transverse plane to the longitudinal axis z and forms a first base 29 of the first chamber K1 and a second base 30 of the second chamber K2 on both sides.

Viewed in the direction of the longitudinal axis z, opposite the partition T or the respective base 29 or 30, the chamber K1 or K2, each delimited on the circumference by a partial section of the peripheral wall 28, is (initially) closed by one of the ceiling parts 27. These ceiling parts 27 are dome-like, further essentially designed in the manner of a hollow hemisphere, each having a radius with respect to the dome-like curved chamber wall 32 in the ceiling part 27, which preferably essentially corresponds to the radius of the middle part 26.

The curved chamber wall 32 of each ceiling part 27 can, as shown, merge into a circular cylindrical wall section 33, which preferably includes the peripheral wall 28 on the central part side. In this regard, a positive connection can be provided between the middle part 26 and each ceiling part 27, for example by engaging a radially inwardly projecting circumferential projection 34 of the wall section 33 in a preferably shape-adapted groove 35 of the middle part 26, which groove 35 runs around the outside of the peripheral wall 28.

Also, in the plane in which the partition T extends, a rib 36 can be formed on the outside of the peripheral wall 28, against which the circumferential end edge of the cylindrical wall section 33 of each ceiling part 27 can come up against a limited stop.

Viewed in the direction of the longitudinal axis z, the cylindrical peripheral wall 28 of the middle part 26 extends essentially into the transition region into the curved, dome-like chamber wall 32 of the ceiling part 27.

In the direction of the longitudinal axis z, there is a length e of the container 1, which can, for example, correspond to approximately 2 to 2.5 times the width f or the container diameter viewed perpendicular to the length e.

In a possible embodiment, the middle part 26 consists of a hard plastic, for example polypropylene. The two ceiling parts 27 can also consist of a gelatin material.

Two chambers K1 and K2 are thus created, each of which is comprehensively delimited by the associated base 29 and 30, the associated section of the cylindrical peripheral wall 28 and the associated dome-like chamber wall 32 of the respective ceiling part 27.

A first substance S1 is accommodated in the first chamber K1 and a second substance S2, which is preferably different from the first substance, is accommodated in the chamber K2. The substances S1 and S2 are preferably powdery. In particular, these are pharmaceutically active substances. These two substances S1 and S2 are to be mixed together in the course of an inhalation process and preferably only at this moment and transferred to the user's respiratory tract via a suction air flow.

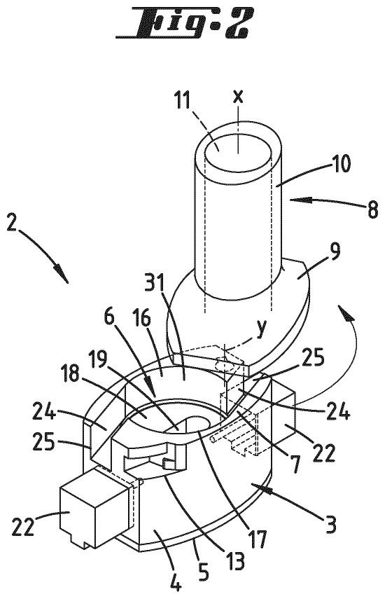

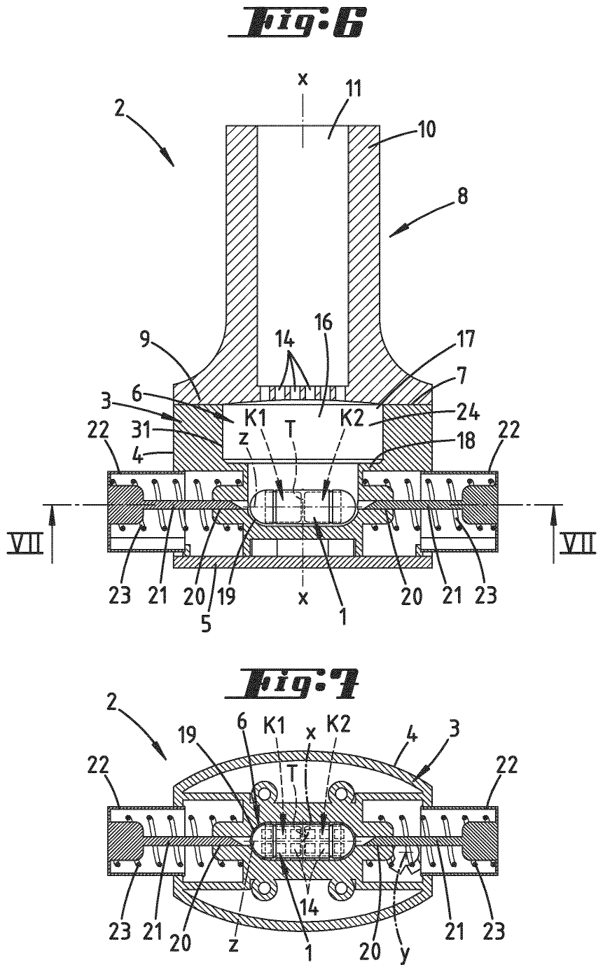

For this purpose, in the chamber open position according to FIG. 2, the container 1 can first be inserted into the recess 19 of the receiving chamber 6, after which the receiving chamber 6 is closed by pivoting the suction section 8 about the pivot axis y. This results in an arrangement as shown in FIGS. 6 and 7.

As can be seen in particular from the cross-sectional illustration in FIG. 7, the recess 19 is preferably provided with a comparatively small excess dimension compared to the container 1 with regard to its length and width. In this regard, a length a and a width b of the recess 19 can be given, which can correspond, for example, to approximately 1.1 to 1.2 times the length e or the width f of the container.

In this position of the container 1, preferably in the recess 19, the piercing needles 21 are actuated by the user by moving the relevant actuation buttons 22 towards one another against the restoring forces of the springs 23 (arrows s).

The piercing needles 21 pass through the container 1 in the area of its ceiling parts 27, resulting in piercing openings 37 in the area of the dome-like curved chamber walls 32. These are essentially and preferably achieved at the zenith of the chamber wall 32, which is curved in a uniform dome-like manner, as a result of the wall being penetrated by the respective piercing needle 21 (compare FIGS. 8 and 9).

Due to limited displacement, the penetration depth of the piercing needles 21 is preferably chosen so that the partition T separating the chambers K1 and K2 is not penetrated.

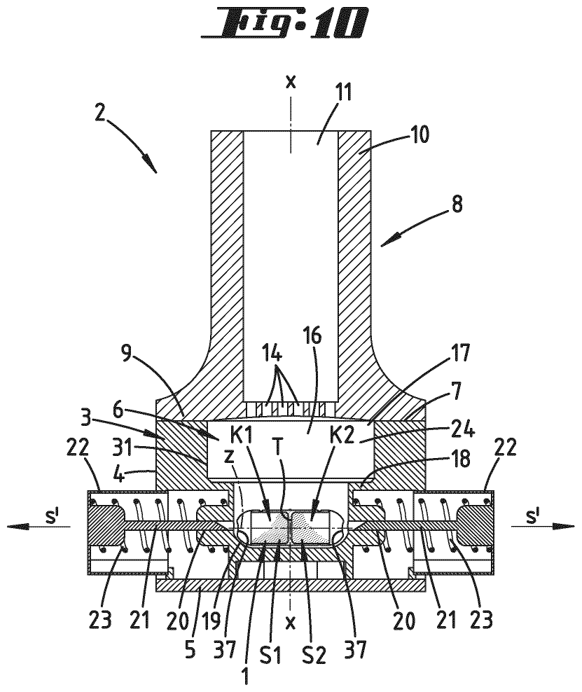

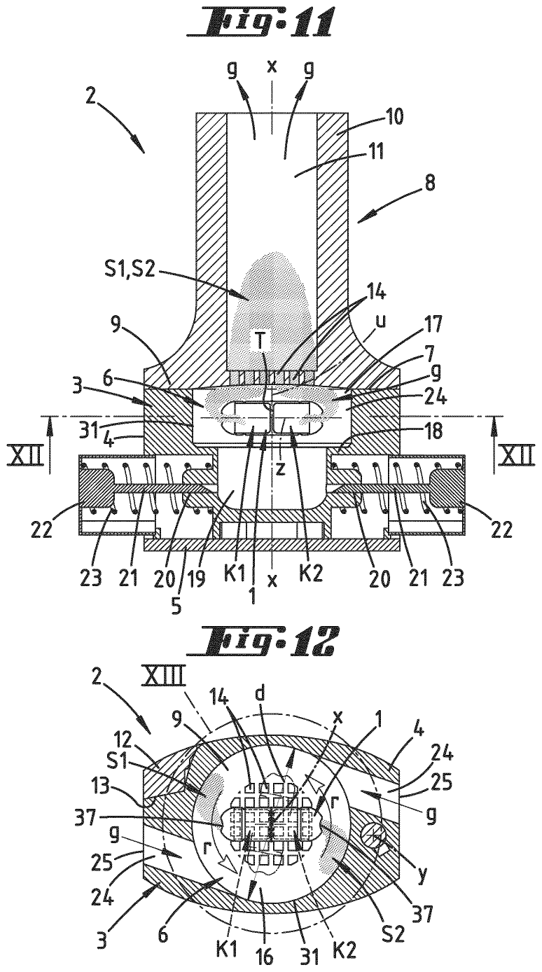

With the repositioning of the piercing needles 21 (arrows s′), which repositioning occurs automatically after the user has canceled the corresponding action on the actuation button 22, the inhalation device 2 with the container 1 is prepared to carry out the inhalation process. The chambers K1 and K2 are each opened by the perforation of the respective ceiling part 27 to dispense the substance S1 and S2 contained in each case (see FIG. 10).

The inhalation process can be opened by enclosing the suction section 8 by the user's lips or, in the case of nasal use, by inserting the suction section 8 into a nostril, whereby a suction air flow (arrow g) is generated through the inhalation device 2 as a result of deep inhalation by the user.

Thereby, air is sucked from the environment through the suction channels 24 and through the openings 14 and the suction channel 11 of the suction section 8 (arrow g). The container 1 is sucked and raised from the area of the recess 19 into the rotation area 16, in which rotation area 16 of the container 1 due to the air flowing essentially tangentially into the rotation area 16 from the suction channels 24 (arrow g in FIGS. 12 and 13), is set in rotation about a rotation axis u which runs essentially transversely to the longitudinal axis z of the container 1 (see arrows r). The housing cover 9 with the openings 14 provided prevents the container 1 raised in this way from entering the suction channel 11.

The axis of rotation u of the container 1 in the rotation area 16 can, as ideally preferred, essentially coincide with the longitudinal axis x passing through the center of the receiving chamber 6, whereby a slightly wobbling superimposition of the rotational movement can also occur in the course of the rotation about the rotation axis u.

As a result of the centrifugal forces resulting from the rotation of the container 1, the substances S1 and S2 are expelled from the chambers K1 and K2 through the piercing openings 37 into the rotation area 16 of the receiving chamber 6, the expulsion being expelled in the radial direction outwards with respect to the axis of rotation u is limited by the peripheral wall 31 of the rotation area 16.

Superimposed by the impulse resulting from the rotation of the container 1 and the air flowing tangentially into the rotation area 16, there is essentially a mixing of the two substances S1 and S2 within the rotation area 16, which supports the resulting effect that the piercing openings 37 of the two chambers K1 and K2 pass through superimposed paths in the course of this rotation of the container 1, so when the container 1 rotates about a position-accurate rotation axis u along a through-path w delimited by the circumferential piercing openings 37 (see FIG. 13).

The rotation of the container 1 in the rotation area 16 can result in a turbulence of the air flow in this area as a whole, so that the air entering the rotation area 16 flows around the container 1 not only essentially in the direction of extension of the chamber-side peripheral wall 31, but also, if necessary superimposed on this flow movement also essentially in the circumferential direction of the container 1, flowing around it at least partially on the outside of the wall along the curved peripheral wall 28.

The chambers K1 and K2 are preferably cleared essentially solely and exclusively as a result of the forces that arise when the container 1 rotates. A flow through the container 1 is prevented by the arrangement of the partition T to form two separate chambers.

This can result in the further positive effect that, due to the dome-like curved inner walls 38 of the wall sections 33 and the centrifugal forces that arise when the container 1 rotates, residues of the first or second substance S1 or S2 are also driven out of the respective chambers K1 and K2, up to, if necessary, complete emptying by directing these substances along the curved wall sections 33 to the piercing opening 37, which is preferably provided in the area of the zenith, for discharge from the chamber (see FIG. 15).

Also in the area of the suction channel 11, suction air g going into the rotation area 16 can result in a suction air vortex with substances S1 and S2 picked up in this suction air vortex and mixed with one another, so that a homogeneous mixture of the two substances reaches the user's airway.

At the end of the inhalation process, the emptied container 1 can be removed from the receiving chamber 6 after the suction section 8 has been moved into the chamber open position. After use, the emptied container 1 lies freely on the floor 18 of the rotation area 16. If necessary, the emptied container 1 can also have fallen back into the recess 19.

List of reference symbols

| | |
|---|---|
| 1 | container |
| 2 | inhalation device |
| 3 | housing |
| 4 | housing wall |
| 5 | housing bottom |
| 6 | receiving chamber |
| 7 | housing edge |
| 8 | suction section |
| 9 | housing cover |
| 10 | spout-like extension |
| 11 | extension |
| 12 | locking extension |
| 13 | niche |
| 14 | opening |
| 15 | cover cap |
| 16 | rotation area |
| 17 | chamber opening |
| 18 | bottom |
| 19 | recess |
| 20 | guide |
| 21 | piercing needle |
| 22 | actuation button |
| 23 | spring |
| 24 | suction channel |
| 25 | suction opening |
| 26 | middle part |
| 27 | ceiling part |
| 28 | peripheral wall |
| 29 | first base |
| 30 | second base |
| 31 | peripheral wall |
| 32 | chamber wall |
| 33 | wall section |
| 34 | projection |
| 35 | groove |
| 36 | rib |
| 37 | piercing opening |
| 38 | inner wall |
| 39 | ceiling area |
| a | length |
| b | width |
| c | depth |
| c′ | depth |
| d | diameter |
| e | length |
| f | width |
| g | arrow |
| r | arrow |
| s | arrow |
| s′ | arrow |
| u | axis of rotation |
| w | passage path |
| x | longitudinal axis |
| y | pivot axis |
| z | longitudinal axis |
| K1 | first chamber |
| K2 | second chamber |
| S1 | first substance |
| S2 | second substance |
| T | partition |

1: A method for mixing a first pharmaceutical powdery substance (S1) and a second pharmaceutical powdery substance (S2) in an inhalation device (2) with which pharmaceutical substance is transported by a suction of air, wherein the first and the second substances (S1, S2) are contained in a container (1) with two separate chambers (K1, K2), wherein the chambers (K1, K2) are arranged one behind the other along a longitudinal axis (z) of the container (1), the method comprising:
in a first step, opening the chambers (K1, K2) of the container (1) by piercing, with forming piercing openings (37), wherein the opening by piercing is carried out with respect to the longitudinal axis (z) on opposite sides, and
in a further step, in the course of suction of air by an user, rotating the container about an axis of rotation (u) in a receiving chamber (6) of the inhalation device (2), which runs transversely to the longitudinal axis (z), whereby the substances (S1, S2) from each chamber (K1, K2) are discharged radially outwards from the container (1) and a mixing of the two substances (S1, S2) thus discharged is achieved.

2: The method according to claim 1, wherein the piercing openings (37) of the chambers (K1, K2) formed by piercing pass through overlapping paths (w) in the course of the rotation of the container (1).

3: The method according to claim 1, wherein, due to the rotation of the container (1), the substances (S1, S2) come against a peripheral wall (16) of the receiving chamber (6) which extends essentially transversely to the axis of rotation (u) being caught thereby.

4: The method according to claim 1, wherein air flowing based on the suction essentially flows around the container (1).

5: The method according to claim 1, wherein the receiving chamber (6) has a bottom (18) with a recess (19) which is essentially adapted to a contour of the container (1).

6: The method according to claim 5, wherein the piercing of the chambers (K1, K2) is carried out when the container (1) is in the recess (19) of the receiving chamber (6).

7: The method according to claim 5, wherein the container (1) is moved out of the recess (19) of the receiving chamber (6) into a rotation area (16) of the receiving chamber (6) adjoining in the direction of flow of the air in the course of a suction of air in the inhalation device (2), in which rotation area (16) the rotation of the container (1) about the axis of rotation (u) is set as a result of the suction.

8: The method according to claim 1, wherein each chamber (K1, K2) has a curved chamber wall (32) located radially on the outside with respect to the chambers (K1, K2), which after piercing the chambers (K1, K2) direct the powdery substances (S1, S2) to the piercing openings (37).

9: Method for delivering a first and a second substance from a container, comprising the steps of:
providing a container (1) with two chambers (K1, K2) comprising a first chamber (K1) and a second chamber (K2), in an inhalation device (2), the chambers (K1, K2) being filled with a first pharmaceutical powdery substance (S1) and a second pharmaceutical powdery substance (S2) respectively, the inhalation device (2) having piercing needles (31) configured for piercing each chamber (K1, K2) of the container (1), the first chamber (K1) having a first base (29) and the second chamber (K2) having a second base (30), the container having a partition (T), the partition (T) separating the first chamber (K1) from the second chamber (K2), wherein the partition (T) forms the first and second base (29, 30) and the first and second chambers (K1, K2) extend in opposite directions to one another, starting from the first and second base (29, 30), respectively, along a common longitudinal axis (z) of the container (1) and form a peripheral wall (28) and a ceiling area (39) opposite the base (29, 30), each ceiling area (39) being concavely curved and being pierceable, and
delivering the first and second substances (S1, S2) from the container (1) by rotating the container (1) about an axis of rotation (u) running transversely to the longitudinal axis (z).

10: The Method according to claim 9, wherein the container (1) has three parts: a middle part (26) which has a partition (T) and at least a partial area of the peripheral wall (28), and two ceiling parts (27) forming the ceiling areas (39).

11: The Method according to claim 10, wherein the ceiling parts (27) consists of a gelatin material.

12: The Method according to claim 10, wherein all of the parts (26, 27) consist of a gelatin material.

13: The Method according to claim 10, wherein all of the parts (26, 27) consist of a hard plastic material.

14: The Method according to claim 10, wherein the middle part (26) consists of a hard plastic material and the ceiling parts (27) consist of a gelatin material.

15: The Method according to claim 9, wherein the container (1) has a cylindrical basic contour with two dome-like ends.

16: The Method according to claim 9, wherein the container (1) has a length (e) given in the direction of the longitudinal axis (z) which is greater than a width (f) of the container (1) given perpendicular thereto.
